(19) United States
(12) Patent Application Publication
(10) Pub. No.: US 2007/0282156 A1
(43) Pub. Date: Dec. 6, 2007

(54) Apparatus For Generating Electric Current Field In The Human Body And Method For The Use Thereof

(76) Inventor: Maurits Konings, Utrecht (NL)

Correspondence Address: KNOBBE MARTENS OLSON & BEAR LLP, 2040 MAIN STREET, FOURTEENTH FLOOR, IRVINE, CA, 92614, US

(21) Appl. No.: 11/629,781

(22) PCT Filed: Jun. 16, 2005
(86) PCT No.: PCT/NL05/00441

§ 371 Date: May 10, 2007

(30) Foreign Application Priority Data
Jun. 16, 2004 (NL) 1026431

(51) Int. Cl. A61N 2/04

(52) U.S. Cl. 600/13

(57) ABSTRACT

The present invention relates to an apparatus for generating an electrical current field in the human body, comprising two or more rotatable magnetic elements for generating of a composite magnetic field, which magnetic elements are positioned such that each magnetic element is rotatable around an axis of rotation; means for producing a rotating motion of the magnetic elements such that upon rotation of one or more of the magnetic elements the composite magnetic field remains substantially constant; and means for disturbing the composite magnetic field, for altering the magnetic field entirely.

Non-invasive electromagnetic neurostimulation has many applications, such as the treatment of neuro-physiological diseases, investigation of function, and pain treatment. For the application of neurostimulation for the sake of pain treatment, the Gate-Control theory (of Melzack and Wall) is important. According to this theory, the transportation of signals along pain-sensitive nerves (C-fibers) can be mitigated by simultaneous stimulation of another type of nerves, viz. the ‘thick fibers’ (T-fibers), which are not pain-related. In this way, pain treatment can be performed using localized and specific stimulation of thick fibers only. This theory is the basis for known pain treatment techniques, such as TENS (Transcutaneous Electrical Nerve Stimulation) and ESES (Epidural Spinal Electrical Stimulation).

The basic principle of electromagnetic neurostimulation is to produce an electric field in neuronal tissue in such a way, that it causes the excitation of neurons.

Prior art techniques that are used to create such an electric field in neuronal tissue, can be divided into two categories:

(i) use of electrodes; and,

(ii) use of magnets (permanent magnets as well as electromagnets).

For both categories, it is known that the electric fields produced with these techniques, are such that, in a finite, bounded, three-dimensional object with homogeneous and isotropic conductivity, the point of maximum field strength is always situated on a location of the boundary of the object, i.e., on the surface between the interior and the exterior of the object. This fundamental limitation is also valid if multiple magnets and electrodes are used. In the following, this fundamental limitation will be referred to as ‘FL’.

The object of the present invention is to avoid the fundamental limitation FL, in order to create the possibility for the occurrence of a local maximum of the intensity of the electrostimulation on a predetermined location inside the tissue, in such a way that the intensity of the stimulation in the skin remains below the intensity that occurs at the location of maximum intensity.

The present invention provides an apparatus for generating of an electrical current field in a human body; this apparatus incorporates:
at least two rotatable magnetic elements for generating of a composite magnetic field, which magnets are positioned in such a way that each magnetic element can be rotated around an axis of rotation;
means to produce a rotating motion of the magnetic elements in such a way that, upon rotation of one or more magnetic elements, the composite magnetic field remains constant; and
means to disturb the composite magnetic field, or alter the magnetic field entirely.

In this apparatus, the “magnetic elements” may contain an electromagnet, a permanent magnet, and/or material that can be magnetized. This magnetizable material can be magnetized by the presence of a non-rotating electromagnet, permanent magnet, or any object of magnetizable material nearby.

The apparatus can produce magnetic fields, in such a way that, by rotating the magnetic elements, electrostatic charge accumulations are produced.

As the magnetic elements rotate in a new and entirely unconventional manner, these electrostatic charge accumulations are produced in spite of the fact that the body is an electric conductor. These charge accumulations do not occur in conventional equipment for magnetic stimulation with stationary electromagnets.

In other conventional methods that do use moving magnets, these charge accumulations barely occur or do not occur at all; if these charge accumulations do occur, the effects of these charge accumulations are dwarfed by the much stronger effect of circular currents that are evoked directly by induction. This will be explained below.

The present invention however contains an apparatus which causes the total magnetic field to remain constant, in spite of the rotation of the magnetic elements.

This can be done by, for instance, having each of the magnetic elements rotate around an axis of rotation that coincides with the axis of symmetry of the (cylinder symmetrical) magnetic field that is produced by each of the magnetic elements.

However, some other embodiments will be described in which the composite magnetic field remains constant during rotation of the magnetic elements whereas the axis of rotation does not coincide with the axis of symmetry of each individual magnet.

As the present invention comprises an apparatus that keeps the composite magnetic field constant in spite of the rotation of the magnetic elements, there will be no flux change in the total magnetic field within the tissues of the body, and hence no circular currents will arise.

Therefore, according to the present invention, the effect of the charge accumulations is no longer dwarfed by the effects of the circular currents, and the effects due to the charge accumulations remain present as useful effects.

As will be explained below (in the section ‘physics background’), the effects of the quasi-static charge accumulations are such that the fundamental limitation FL can be avoided, provided that the charge accumulations are used in a proper way, as is done in the case of the invention at hand. This enables the creation of a local maximum of the intensity of the electrostimulation on a deliberately chosen spot inside the tissues, in such a way that the intensity of the stimulation in the skin remains below the intensity that occurs at the location of maximum intensity.

In the case of the present invention, means for the sudden disturbance of the total magnetic field is used, in such a way that the invention at hand is capable, in a surprising way, of using the shifting of the quasi-static charge accumulations to create the desired current field.

These means for disturbance may comprise means to reverse the polarity of the magnetic field, and do so with a frequency that will be referred to as the ‘reversion frequency’.

In some preferred embodiments that will be described below, a reversal frequency will be used that is smaller than the frequency of rotation. The reversal frequency is below 3000 Hz in that case, and preferably even lower than 1000 Hz.

In yet another preferred embodiment, the desired constant volume of the total magnetic field is achieved by using magnetic elements that have an anisotropic magnetizability, such as in the case of e.g. ‘shape anisotropy’ or ‘crystalline anisotropy’.

In order to achieve high rotational velocities of the magnetic elements, in one of the preferred embodiments air bearings are used. In another preferred embodiment air thrust or air pressure is used to cause rotation, in combination with air bearings or other bearings.

Other advantages and features of the present invention will be clarified using the figures in this document, in which:

FIG. 1: shows a desirable situation of neuronal stimulation, to be realized by using the present invention;

FIG. 2: shows a known technique to produce a significant change of flux, as function of time, inside the tissue using a rotating magnet;

FIG. 3: shows a diagrammatical view of the effect of an imposed electromotive field E_{ROT};

FIG. 4: shows a diagrammatical view of the effect of an imposed electromotive field E_{DIV};

FIG. 5: shows a diagrammatical view of the essentials of the present invention;

FIG. 6: shows diagrammatical graphs to clarify the additional feature ‘combination’;

FIG. 7: shows a sectional side view of one of the magnetic elements out of the three magnetic elements that are used in a first preferred embodiment of an apparatus according to the present invention in a first situation of use.

FIG. 8: shows a perspective view of the apparatus from FIG. 7;

FIG. 9: shows a perspective view of a first preferred embodiment of an apparatus according to the invention at hand;

FIG. 10: shows a diagrammatical view of the apparatus from FIG. 9, as sectional side view;

FIG. 11: shows a diagrammatical view of the apparatus from FIG. 9, viewed in a different sectional side view, viz. a transversal cross-section, in which the view direction is parallel to the skin, and parallel to the spinal cord;

FIG. 12: shows a diagrammatical side view of another preferred embodiment of the apparatus according to the present invention, according to the principle of the ‘feeding magnet’; and

FIG. 13: shows a diagrammatical side view of yet another preferred embodiment of the apparatus according to the present invention, in which the principle of the ‘feeding magnet’ is used, and furthermore magnetic elements are used that are composed of oblong objects of magnetizable material, in which each of these objects has anisotropic magnetizability due to ‘shape anisotropy’.

### EXPLANATION ACCOMPANYING THE FIGURES

FIG. 1: Diagram of the desired shape of a graph 3 showing the strength of the electric neurostimulation field Eₙₑᵤᵣₒₛₜᵢₘ (along the vertical axis 1), as a function of the distance z to the skin. The three-dimensional spatial position x as x=(x,y,z), x and y are kept constant, and z is the variable running along the horizontal axis 2. Inside the region S to be stimulated, the graph is located between the threshold value of the thick fibers E^{T}_{threshold} (indicated by the interrupted line 4), and the value of the C-fibers E^{C}_{threshold} (indicated by the interrupted line 5). Within the region S the following formula therefore holds:
E^{T}_{threshold}<Eₙₑᵤᵣₒₛₜᵢₘ(x,y,z)<E^{C}_{threshold}

The position of the skin is indicated by the vertical interrupted line 6. At the location of the skin, no neurostimulation takes place. On all positions outside the region S, no neurostimulation takes place either, so:

For all x∈LΛx∉S:Eₙₑᵤᵣₒₛₜᵢₘ(x)<E^{T}_{threshold}<E^{C}_{threshold}

FIG. 2: Diagram of a known technique in which a change of flux is being caused inside the tissue 9 by the rotation of a magnet. In this particular case, a bar magnet 7 is depicted, having a north pole N and a south pole S. The magnet causes a magnetic field that is approximately cylinder-symmetrical around the axis of symmetry 11. In the case of this known technique, the magnet rotated around an axis of rotation 10 that is perpendicular to the plane of the drawing, as is indicated by a point. The skin 8 is located at the boundary of the tissue 9; the magnet is located outside the body.

FIG. 3: Diagram of the effect of an imposed, purely rotational, electromotive field E_{ROT}, in a volume of tissue 12, with the skin 14. Since ∇·E_{ROT}=0, no charge accumulations arise. Circular currents (eddy currents) do arise however, of which the maximum 13 is located near the skin.

FIG. 4: Diagram of the effect of an imposed, purely divergent, electromotive field E_{DIV}, in a volume of tissue 12, with the skin 14. Since ∇×E_{DIV}=0, no circular currents arise. Charge accumulations do arise however; in this figure, the positive and negative charge accumulations are indicated by 15 en 16, respectively.

FIG. 5: Diagram of the situation in which a magnetic object 19 rotates as indicated by the rotation vector 18 (this rotation vector Ω indicates the direction of the axis of rotation), and also in which the magnetic object produces a cylinder-symmetrical pattern of magnetic field lines 20, of which the axis of symmetry 17 (indicated by the interrupted line) coincides with the axis of rotation. This situation represents the principle underlying a possible, simple, embodiment of the invention. In order to make this simple embodiment complete, the addition of one extra rotating magnetic object is needed, as explained in the text in the section with the caption ‘combination’.

FIG. 6: Diagrammatical graphs for clarification of the additional feature ‘combination’. In the graph at the left 21, the value of ρm(x) (which is the charge density, in Coulomb per m³, along the vertical axis 23) is displayed as a function of the distance z to the skin for constant values of x and y (with x=(x,y,z)), for various magnets m. The location of the skin is indicated by the vertical interrupted line 25. The interrupted curved line 26 indicates the charge accumulation ρM1(z) resulting from a small magnet M1 rotating with an angular frequency ω1. The magnet M1 is located close to the skin. The interrupted curved line 27 indicates the charge accumulation ρM1(z) resulting from a much larger magnet M2 rotating with an angular frequency ω2, with ω2=ω1. The magnet M2 is located at a larger distance to the skin. Because of the larger size of magnet M2, the interrupted curved line 27 is declining less steeply in comparison to the interrupted curved line 26. Furthermore, at the location of the skin, curve 26 is much steeper than curve 27, because magnet M2 is located further away from the skin. The larger distance from M2 to the skin entails furthermore that, near the skin 25, the value of ρM1 is much larger than ρM2, in spite of the fact that M2 is much larger in size than M1. An essential point of the additional feature ‘combination’ now is that the speed of rotation of M2 can be increased in such a way (reaching an angular frequency ω3, with ω3>ω2), that the resulting charge distribution ρM3 (indicated by the curve 28) now equals ρM1 at the skin. By comparing curve 26 with curve 28 of the ρM3 that results from the now faster rotating larger magnet M2, it can be seen that

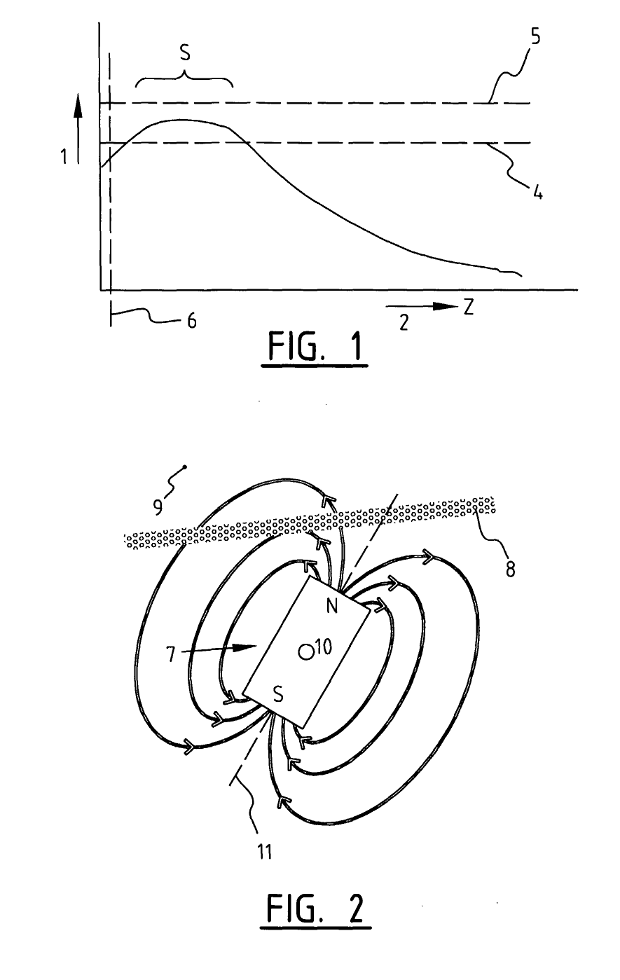

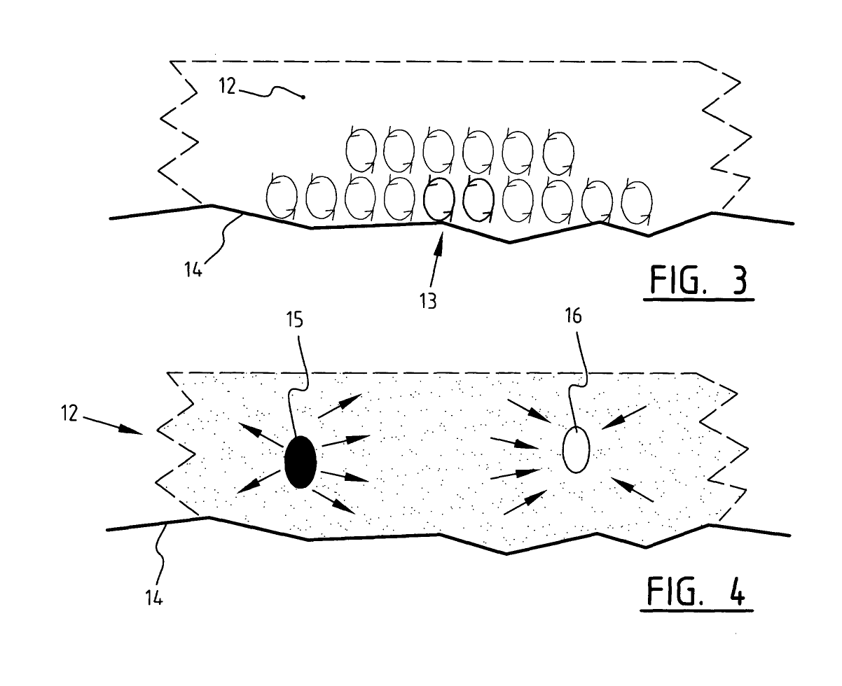

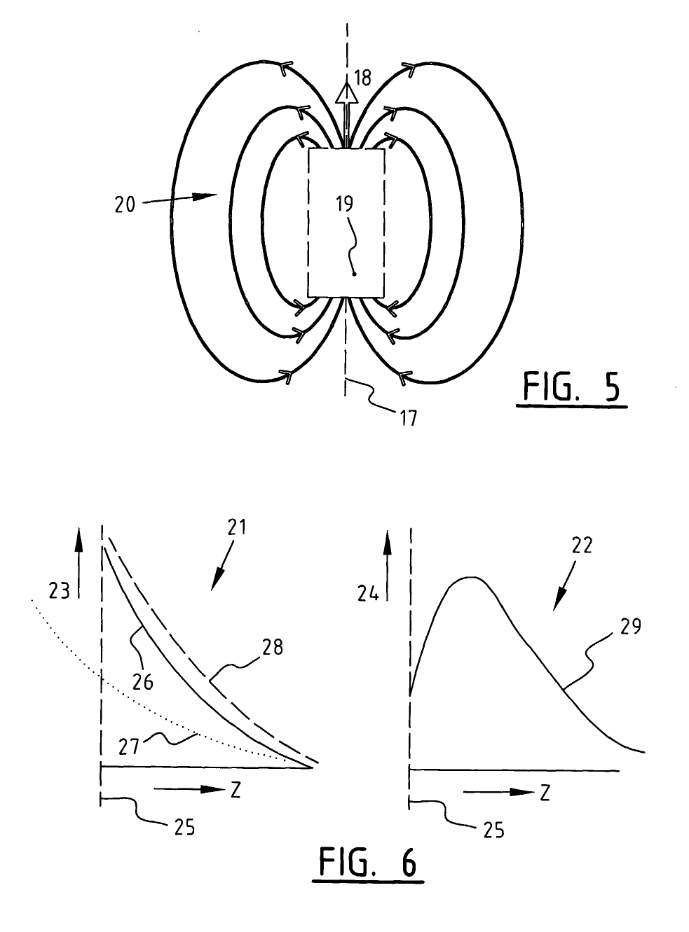

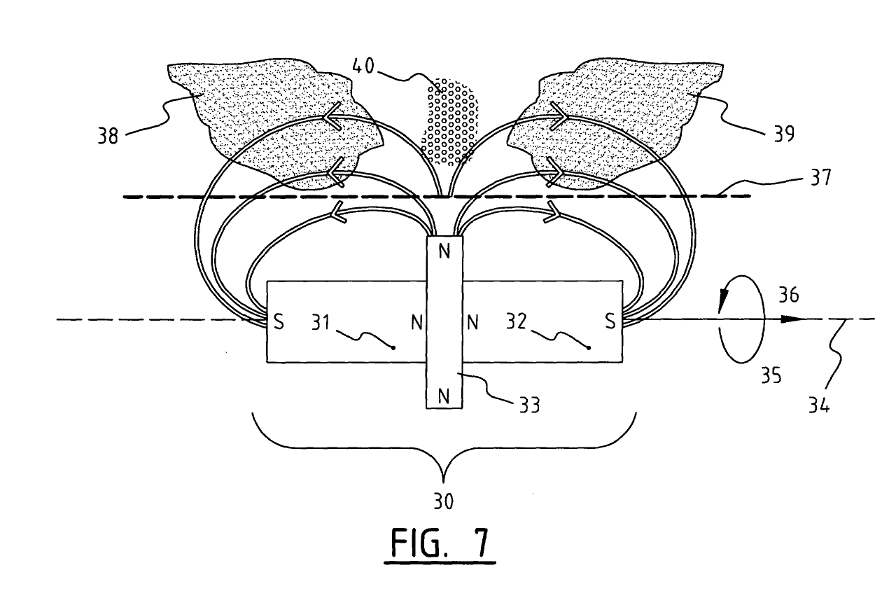

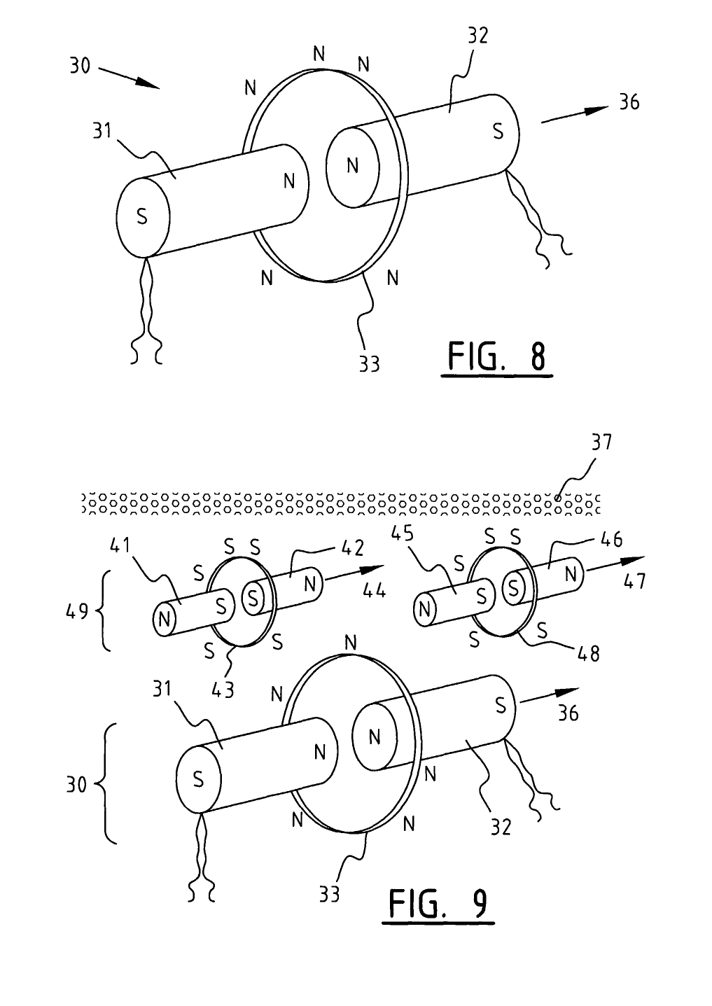

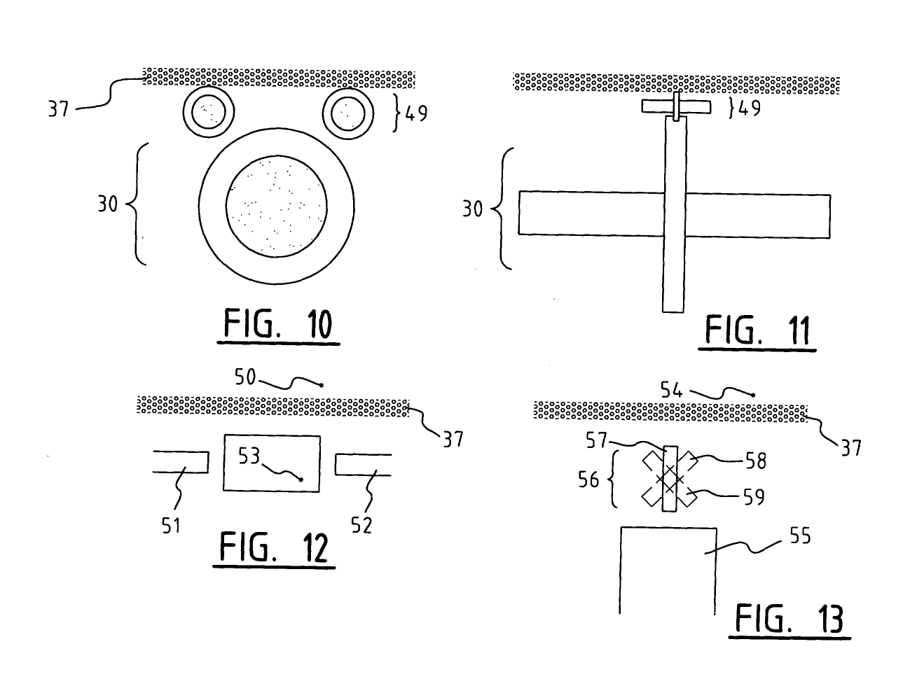

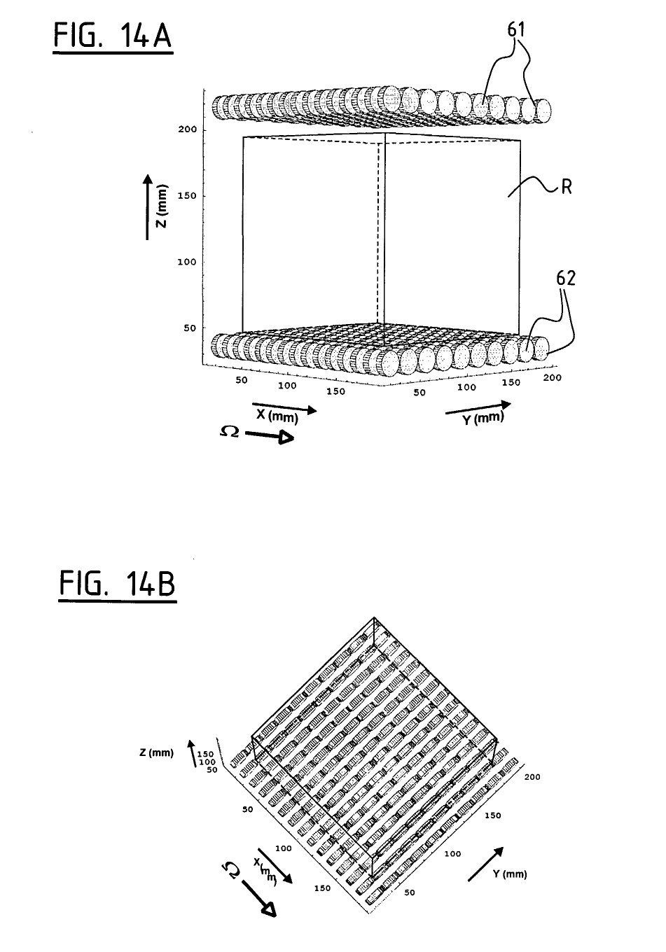

ρM3(z_{SKIN})−ρM1(z_{SKIN})=0, and ρM3(∞)−ρM1(∞)=0, and ρM3(z_{TISSUE})−ρM1(z_{TISSUE})>0, in which z_{SKIN} is located in the skin, and z_{TISSUE} inside the inner tissue (z_{SKIN}<z_{TISSUE}).

The linear combination ρM3(z)−ρM1(z) can be obtained by having:

either magnet M1 spin in a direction opposite in comparison to M2; or

by having the polarity of the magnetic field from M1 and M2 be oppositely directed.

The curve 29 in graph 22 of the linear combination ρM3(z)−ρM1(z) therefore shows a maximum value that is not located in the skin 25, but at a location deeper inside the tissues. The scales of the vertical axes 23 and 24 are different. The physical quantities that are represented along the axes 23 and 24 are identical. Graph 22 therefore illustrates the fact that the desired situation, as indicated in FIG. 1, can be obtained by using the invention.

FIG. 7: A sectional side view of one of the magnetic elements out of the 3 magnetic elements that are used in a first preferred embodiment of an apparatus according to the invention at hand in a first working state. The magnetic element 30 is positioned in such a way that it can rotate (spin) around the axis of rotation 34. This magnetic element 30 is composed of a first cylinder-shaped magnet 31, a second cylinder shaped magnet 32, and a cylinder-shaped disk 33 of magnetizable material, which is located between the magnets 31 and 32. At each moment in time, the polarity of each of the two magnets (31 and 32) is such that these two magnets (31 and 32) exert a repelling force on each other. This implies that, either: the north pole of magnet 31 as well as the north pole of magnet 32 touch the magnetizable disk 33, or that the south pole of magnet 31 as well as the south pole of magnet 32 touch the magnetizable disk 33, depending on the moment in time. The choice of the moment in time is chosen arbitrarily in this figure. As a result (i.e., as a result of the fact that the north pole of magnet 31 as well as the north pole of magnet 32 touch the magnetizable disk 33), there is, magnetically speaking, a ‘north pole’ on each spot on the curved outer surface of the magnetizable disk 33. Because of the cylinder-symmetry of each of the components of the magnetic element 30, and because of the fact that the axes of symmetry of each of the components 31, 32, and 33 coincide with the axis 34, the magnetic field of magnetic element 30 is cylinder symmetrical as well; its axis of symmetry coincides with 34. An important fact is that the axis of symmetry 34 coincides with the axis of rotation 36. As a result, rotation of the magnetic element 30 around the axis of rotation 36 does not produce any changes in the magnetic field, and hence does not evoke circular currents in the human body nearby. In an example of an application that is shown schematically in FIG. 7, the magnetic element 30 is located in the vicinity of the spine 40. The magnetic field lines originating from the magnetic element 30 penetrate through the skin 37 into the deeper tissues, such as the dorsal muscles 38 and 39 that are located at each side of the spine 40. FIG. 7 shows a cross-section through the human body; the view direction is parallel to the skin in the direction from the head to the feet. The situation as has been depicted in FIG. 7 causes the formation of charge accumulations in the dorsal muscles 38 and 39, in according to the formula:
ρ(x)=2ε(x)(B(x)·Ω),
in which ρ(x) is the charge density at position x, ε(x) is the dielectric permittivity at position x,B(x) is the magnetic field at position x that originates from rotating sources, Ω is the rotation vector of the magnetic element or magnetic elements. Furthermore, the sign (positive or negative) of the charge accumulation in muscle tissue 38 is the opposite of the sign (negative or positive) of the charge accumulation in muscle tissue 39, which is desirable, and which is caused by the fact that the magnetic field in 38 is mirrored with respect to the magnetic field in 39. If, subsequently, by using a pole-reversal apparatus, all magnetic poles in 30 are changed into their corresponding opposite poles, an exchange of electric charge will take place between the muscle tissues 38 and 39. This causes the existence of an electric current field, for example at the site of the dorsal ganglia that are located between the dorsal muscles 38 and 39, in the direct neighborhood of the spine 40. This causes the desired neurostimulation at the site of the dorsal ganglia.

FIG. 8: A diagrammatical view of the apparatus from FIG. 7.

FIG. 9: A diagrammatical view of the three magnetic elements that are used in a first preferred embodiment of an apparatus according to the present invention in a first working state. The magnetic element 30, which is shown in the FIGS. 7 and 8 as well, is located at a larger distance from the skin 37 in comparison to the other two magnetic elements (49). Furthermore, the magnetic element 30 is larger than the other two magnetic elements, and has a stronger magnetic field, in order to make the additional feature ‘combination’ work (as will be explained below the heading ‘further explanations’). The cylinder-shaped disks 33, 43, and 48 are all located within one single plane. Except for the dimensions, each of the two smaller magnetic elements (49) has all the properties to match the description of magnetic element 30, as given for FIG. 7. Of each magnetic element, its axis of symmetry coincides with its axis of rotation.

FIG. 10: Diagrammatical view of an apparatus from FIG. 9, this time rendered as a sectional side view. This is a sagittal cross-section, with a side view that is parallel to the skin, but perpendicular to the spine.

FIG. 11: Diagrammatical view of an apparatus from FIG. 9, this time rendered as another sectoral side view. This is a cross-section, with a side view that is parallel to the skin, and parallel to the spine.

FIG. 12: shows a diagrammatical side view of another preferred embodiment of the apparatus according to the present invention, according to the principle of the ‘feeding magnet’ (as will be explained below the heading ‘further explanations’). The magnetizable object 53 is rotating around its longitudinal axis, and is located between the non-rotating poles 51 and 52. The magnetic poles 51 and 52 are either both a north pole, or they are both a south pole. this causes the formation of a pattern of magnetic field lines inside the body 50 that is comparable to the pattern of magnetic field lines in FIG. 7.

FIG. 13: shows a diagrammatical side view of yet another preferred embodiment of the apparatus according to the invention at hand, in which the principle of the ‘feeding magnet’ is used, and furthermore a magnetic element 56 is used that is composed of small oblong objects of magnetizable material (only three small oblong objects (57, 58, 59) are shown here), in which each of these objects has anisotropic magnetizability due to ‘shape anisotropy’. These small objects are being magnetized by the feeding magnet 55. In this way, it is possible to obtain a magnetic field that is almost constant inside the body 54, in spite of the fact that the axis of rotation of the compound magnet is perpendicular to the length axis of each individual oblong small object.

FIG. 14: a DeepFocus neurostimulator set-up in a ‘sandwich’ configuration, together with homogeneous cube R. In this figure, only the rotating parts of the neurostimulator are shown, i.e., 150 soft ferromagnetic cylinder-shaped magnetic elements 61, resp. 62 are shown at each of two opposing sides of the cube R.

The cube R is indicated by thick lines. The direction of the rotation Ω vector of each rotating magnetic element coincides with the x-axis.

14a: side view; 14b: top view. For the purpose of visibility, the top layer of magnets is not shown in FIG. 14b.

FIG. 15: a DeepFocus neurostimulator set-up in a single-sided configuration, together with a homogeneous oblong rectangular volume Rblock 75. Only the rotating parts 77, 78 of the neurostimulator are shown. The oblong volume Rblock is indicated by thick lines.

Again, the direction of the rotation vector Ω of each of the rotation magnetic elements coincides with the x-axis. (a): side view; (b): top view.

FIG. 16: Application of a preferred embodiment of the DeepFocus Neurostimulator to fight pain in the back.

16a: sketch, showing a patient from the back side, and showing rotating magnetic elements 81, placed on the back side of a patient in the vicinity of the spines.

16b: sketch of a transversal cross-section of a patient, showing the tenth thoracic vertebra (T10), and dorsal muscles (M).

16c: diagram of the charge accumulations in a cross-section of the patient (the same cross-section as in 16b), at some instance in time, not during a polar reversal event.

FIG. 17: field lines and contours of the magnetic field B_{onewheel} of one single wheel of soft iron inside the homogeneous cube R 17a, and below the cube R 17b, when subjected to an externally applied field B_{applied} of 2 Tesla. In this figure, the magnetic field is shown after subtraction of the applied field of 2 T. The horizontal line separating part 17a of the figure from part 17b denotes the bottom surface of the cube R. The distance between the bottom surface of R and the rounded outer surface of the single wheel is 13.5 mm. The wheel is viewed from the side; the flat surfaces of the wheel are perpendicular to the (x,z)-plane.

17a: contour plot of Bₚₐᵣ(x) on a (x,z)-plane near the single rotating wheel as a contour plot. The values printed near the contours are in mT. The (x,z)-plane runs through the center of the single wheel.

17b: arrows indicating the direction of the B_{onewheel}.

### Further Explanations

Given the fact that human tissues are electrical conductors, every electric (or electromotive) field E_{applied}(x) that is imposed on the tissues from outside the tissues, will immediately give rise to an electric current density distribution J(x), in which x is a three-dimensional vector indicating a three-dimensional position. (Typographical detail: the underlining of x serves to indicate that x is a three-dimensional vector). The exact values of J(x) as a function of the position x depend on the specific electric conductivity distribution σ(x). Calculation of the J(x) distribution on basis of the given E_{applied}(x) en σ(x) distributions is, generally speaking, a complicated non-linear problem. If however the J(x) distribution is known for all positions x inside the conducting body, the local Eₙₑᵤᵣₒₛₜᵢₘ(x) that is experienced by neurons on a location x, is imply equal to:
Eₙₑᵤᵣₒₛₜᵢₘ(x)=J(x)/σ(x)

In many situations in which neurostimulation is used, the following situation is desired: within a small volume S inside the human body L, one single specific type of fibers (e.g., the thick fibers) need to be stimulated, whereas the pain nerves, i.e. the C-fibers, are not to be stimulated at all. Furthermore, outside the specific region S, no fibers of any type should be stimulated at all. This implies that even neurons inside the skin near the equipment outside the body should not be stimulated. In general, stimulation (activation) of a neuron occurs if, at the location of the neuron, the strength Eₙₑᵤᵣₒₛₜᵢₘ of the local Eₙₑᵤᵣₒₛₜᵢₘ field exceeds a certain threshold level (the dependency on the orientation of the local Eₙₑᵤᵣₒₛₜᵢₘ field is not taken into consideration here). The height of the threshold value depends on the fiber type. The threshold value E^{C}_{threshold} of the neurons inside C-fibers, is higher than the threshold value E^{T}_{threshold} of the neurons inside thick fibers. Therefore, it is possible to choose the value of Eₙₑᵤᵣₒₛₜᵢₘ in such a way that the thick fibers are activated, whereas the C-fibers are not activated. In summary, the desired situation in which inside the region S only the thick fibers are stimulated, and outside S no fibers are stimulated at all, is reached if:
For all x∈S:E^{T}_{threshold}<Eₙₑᵤᵣₒₛₜᵢₘ(x)<E^{C}_{threshold}
and !
For all x∈LΛX∉S:Eₙₑᵤᵣₒₛₜᵢₘ(x)<E^{T}_{threshold}<E^{C}_{threshold}

In FIG. 1, a sketch of the desired situation is shown.

Limitations of Known Techniques

The use of electrodes on the skin is a simple, but not very effective way to achieve neurostimulation in tissues that are located deep inside the body, because the electrostimulation in the tissue caused by the electrodes is such that the point of maximum current density is located in the immediate vicinity of the electrodes, i.e.: near the skin, whereas in the other tissues, in case of a completely homogeneous conductivity distribution, the current distribution is spread out without any maxima or minima deeper inside the tissue.

Invasive (needle-based) electrodes, such as in the case of ESES (Epidural Spinal Electrical Stimulation), are much more effective, and, evidently, spatially selective because the invasive electrodes are positioned in the immediate vicinity of the neuronal tissue to be stimulated. Since however the invasive nature of the invasive electrodes has many serious disadvantages, the need for non-invasive, spatially selective stimulation of deeper tissues is still remaining.

This patent application is a consequence of this remaining need.

Another known technique for electrostimulation uses magnets. The easiest way to create electrical current with a magnet is to create a situation in which the strength or direction of the magnetic field oscillates as a function of time at the location where electrostimulation is desired. Up to now, all known techniques that are described in public documents are based on the basic principle of the oscillating magnetic field.

The techniques that are described in literature can be divided into two categories:

(1) Techniques using electromagnets (one, or more than one, electromagnets) that are fed with an alternating current flowing through the coil. The magnets are all stationary, and are all located on fixed positions with respect to the tissues to be stimulated. The electromagnets thus do not rotate or translate, but are motionless; and

(2) Techniques using rotating or vibrating electromagnets, or electromagnets that are in motion with respect to the tissues in another way. In this case, in all known techniques, the motion of the magnets is such that significant changes in magnetic flux arise in the tissues as function of time. In all known techniques, the purpose of the motion of the magnets is to produce significant changes in magnetic flux in the tissues as function of time.

In the following, it will be explained that both existing techniques ((1) and (2), described directly above) are not suited to create a region of maximum stimulation that is located deeper inside the tissues.

Limiting Factor: the ‘Maximum Principle’

For known techniques as described under (1) (see directly above), which use fixed, motionless magnets that do not rotate or translate with respect to the tissue, the fact that these known techniques are not suited to create a region of maximum stimulation that is located deeper inside the tissues, follows from a limitation L that is a direct consequence of the mathematical principle called the ‘Maximum Principle’, as is explained directly below.

The Maximum Principle is a mathematical principle associated with the Laplace equation.

The maximum principle reads: If inside a homogeneous, finite three-dimensional body R, having a constant, homogeneous and isotropic specific conductivity, the Laplace equation (∇²E=0) holds, then the maximum of (each component of the) E-field is located somewhere on the boundary of R, and never inside R.

The limitation L (see above) now reads: If in a homogeneous, finite three-dimensional body R, having a constant, homogeneous and isotropic specific conductivity, a electrostimulation field E^{fix}_{applied} is produced by an electromagnet (or many electromagnets) using low frequency AC currents or DC current in the coils, in such a way that all magnets and electromagnets are located outside the body R, and furthermore, the electromagnets and magnets are all motionless, and hence do not rotate or translate or vibrate, then: inside R, the Laplace equation holds, and, as a result, the maximum of |E^{fix}_{applied}| will always be located somewhere on the boundary of R (i.e.: somewhere on the two-dimensional boundary surface on the outside of R), and never inside R.

The fact that the limitation L holds for all presently publicly known techniques using motionless magnets for non-invasive electrostimulation, can be shown on basis of the Maxwell equations.

The known techniques of the second category (2), (see above), which are designed to create a change of magnetic flux as function of time using rotating magnets, all have the common feature that the intended creation of flux variation as function of time is only possible if the line connecting the magnetic poles of each magnet does NOT coincide with its axis of rotation. In this case, the ‘axis of rotation’ means the axis (in space) which is the axis of rotation of an actual, physical, rotational motion of the matter that constitutes the material of which the magnet is made.

Of many magnets used today, the magnetic field is approximately cylinder-symmetrical. For the intended creation of flux variation as function of time, according to the known techniques (2), the axis of symmetry should NOT coincide with the axis of rotation.

Summary of the Present Invention

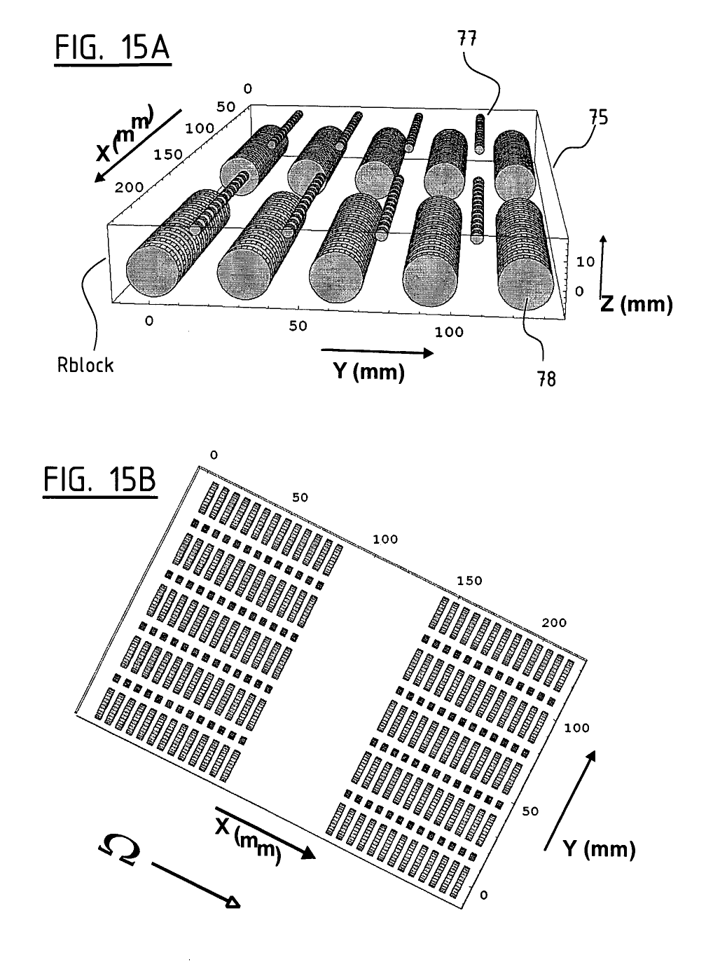

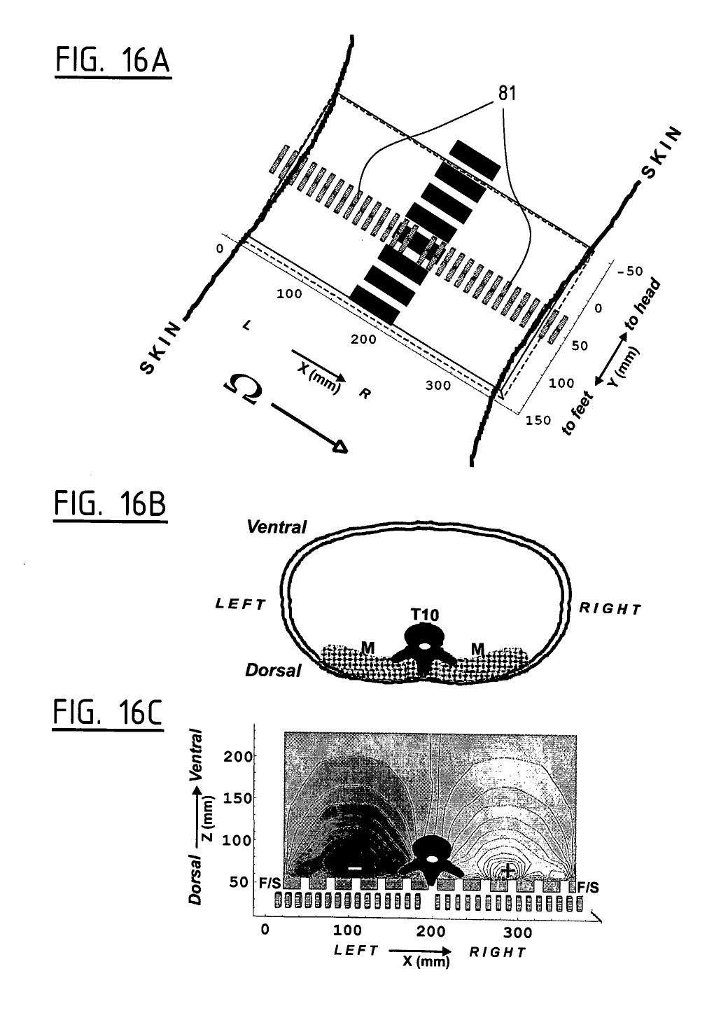

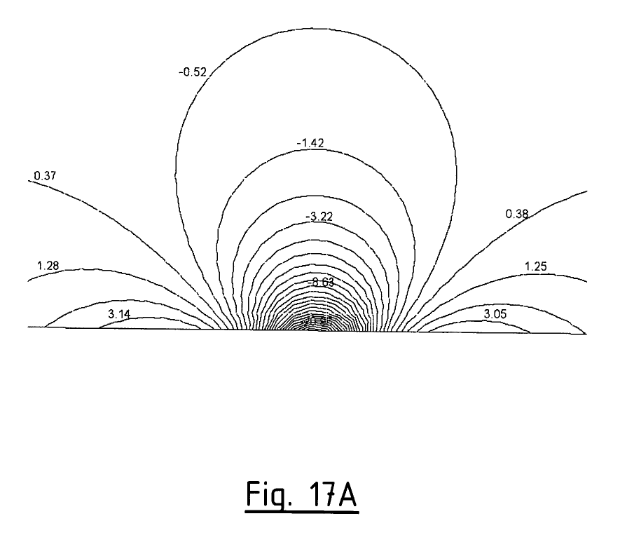

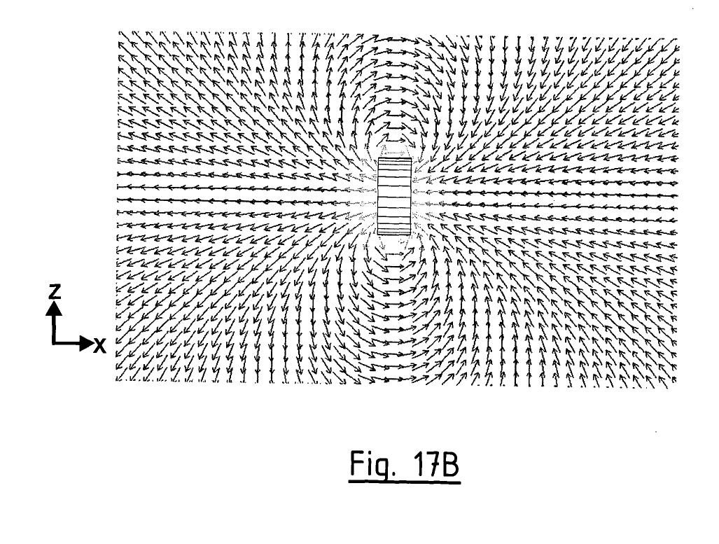

This patent application relates to the idea that non-invasive spatially selective local neurostimulation in regions deeper inside the body DOES become possible if the rotating magnets are applied and used in a way that is fundamentally different from the way they are used up to now in known techniques. In the case of the present invention, and, consequently also in the case of a preferred embodiment of the invention, the magnets are rotated in such a way that the divergence of the Lorentz force field, and hence the charge accumulation, is maximized, and, at the same time, rotated in such a way that the curl of the Lorentz force field, and hence the circular currents and the eddy currents, are reduced to zero. The absence of circular currents and eddy currents is guaranteed by the fact that during the rotation of the magnetic elements, the flux of the magnetic field does not change. In other words: The absence of circular currents and eddy currents is guaranteed by the fact that during the rotation of the magnetic elements, the magnetic field vector B(x) remains constant for each point x in the body R, in spite of the rotation of the magnetic elements. This can be achieved, for example, by letting the axis of rotation of each magnetic element coincide with its axis of cylinder symmetry. (see FIG. 5).

Principle of Operation of the Present Invention

The principle of operation is based on the idea as described above under the heading ‘summary of the present invention’, and therefore is characterized in the fact that Lorentz forces (v×B) are acting on the electrical charges inside the tissue, whereas the magnetic field B(x) remains constant during the rotation of the magnetic elements. As a result, charge accumulations arise inside R. In order to let this phenomenon be useful for the purpose of the creation of a local maximum of the current density inside R, two additional features (‘shift’ and ‘combination’) are needed:

Additional Feature ‘Shift’:

As explained above, the charge accumulations are caused by the rotation of the magnetic elements whereas B(x) remains constant for each position x inside R. If it is desired however that these charge accumulations cause a current density field J(x,t) however, so that a neurostimulation electric field Eₙₑᵤᵣₒₛₜᵢₘ(x,t)=J(x,t)/σ(x) arises, then it is necessary that these charge accumulations start migrating through the tissues, because electric current is by definition the displacement of electric charges.

This migration of the charge accumulations can be achieved by, for example, reversing the polarity of all magnetic poles suddenly at some point in time t. In one of the preferred embodiments, to be described below, the rotating cylinder-shaped magnetic elements are magnetized by non-rotating electromagnets placed nearby. The quasi-static magnetic field that is produced by every electromagnet, is produced by a current that runs through a coil of conducting wire. The rotating cylinder-shaped magnetic element may be placed inside the coil, so that it functions as the soft-iron core of the electromagnet. Each magnetic rotating element rotates in such a way that its axis of symmetry coincides with its axis of rotation.

The current that runs through the coil is step-wise constant, i.e.: its intensity as function of time follows a ‘square wave’-like graph, i.e.: the current is constant unless, within a short time interval, an ‘event’ takes place, i.e., a sudden alteration of the magnetic field. This sudden alteration in the magnetic field causes sudden displacement of the charge accumulations, and corresponds to the ‘jumps’ in the square wave graph describing the intensity of the current running through the coil as function of time. During such an ‘event’, or ‘jump’ in the graph of the current as function of time, all magnetic poles of all the magnets are suddenly reversed, which is accomplished by multiplying the feeding currents in the coils with −1 (‘minus one’), i.e.: by means of sudden manipulation of the voltage source or current source which creates the current running through the coils, the direction of the currents that run through the coils is suddenly reversed. The frequency f_{REVERSAL} with which these sudden reversals of polarity takes place during the course of time, determines the frequency of the ‘events’ (the ‘spikes’ in the value of |Eₙₑᵤᵣₒₛₜᵢₘ|) which causes the neurostimulation.

Therefore, there are in fact two frequencies that play a role in the invention: the angular frequency ω (which is the angular frequency with which a magnetic element rotates around its axis of symmetry), and the frequency f_{REVERSAL} (which is the number of reversal events per unit time). The value of W needs to be high enough to give rise to significant charge accumulations, whereas the frequency of ‘reversal events’ f_{REVERSAL} should be low enough to stay within the frequency region within which neurons are still sensitive to stimulation. Generally speaking, the following inequality will therefore hold:
f_{REVERSAL}<<ω

Additional Feature ‘Combination’:

Using the methods and features of the invention described above, it is no longer fundamentally impossible to create a maximum in the stimulation field that is located somewhere deep inside the tissue, because the fundamental limitation FL is not valid in the case of the present invention. Another additional feature of the invention is still needed to actually be able to gain from the absence of the fundamental limitation FL, and to actually create such a maximum deep inside the tissues.

In order to obtain this situation, multiple rotating magnetic elements need to be used (instead of merely 1), in such a way that, by superposition of the Lorentz forces, a total charge density distribution is created that during a reversal ‘event’ the desired maximum intensity deep inside the tissue is realized. The rotating magnetic elements may be electromagnets or merely magnetizable objects.

In all cases, the total magnetic field B(x) should remain constant as function of time (except for the small time intervals during which the reversal ‘events’ take place), in spite of the rotating motion of the magnetic elements.

In order to obtain the desired total charge distribution, the phenomenon is used that for each rotating magnetic element, there are two degrees of freedom: its angular frequency of rotation, and its magnetic field strength. In combination with the fact that the magnetic elements can be positioned at various distances to the skin, a set of different ‘steepnesses’ of the decay of the contribution to the total charge distribution can be obtained as function of the distance to the skin.

A main aspect of the additional feature ‘combination’ now is that, by using a combination of different magnetic elements, of different sizes, rotating at different speeds, and positioned at different distances to the skin, the superposition of the charge accumulation effects can be such that the desired current density distribution is obtained during the pole reversal events.

### PREFERRED EMBODIMENTS

A number of preferred embodiments of the present invention are possible, in which the number of magnets and the configurations of magnets vary, as well as the ways in which the fast rotation of the magnets is achieved. In the following, indicated by the label ‘(a)’, a simple preferred embodiment is described; and, subsequently, indicated by labels ‘(b)’ and ‘(c)’, more advanced methods to obtain fast rotation of the magnetic elements are described.

(a) A simple embodiment uses at least two rotating magnetic elements (see FIG. 9), and uses preferably three magnetic elements. As an example, an apparatus is described here using two magnetic elements, indicated by M1 and M2, respectively. Both magnetic elements are equal in shapes and design; the essential differences between M1 and M2 are that M1 is much larger than M2, and that M1 rotates faster than M2, and that M2 is located directly near the skin, whereas M1 is located at a larger distance to the skin. Furthermore, either the polarity of the magnetic field M2 is opposed to that of M1, or the direction of rotation of M2 is exactly opposed to that of M1. This enables the situation in which the charge accumulation r_{M1} in the skin, due to the rotation of M1, is opposite in sign with respect to the charge accumulation r_{M2} in the skin due to the rotation of M2. As a result, the desired situation can be achieved in which almost no electric current is running through and near the skin, during the reversal of polarity of the magnets.

Furthermore: resulting from the different slopes with which the magnetic fields from M1 and M2 decay within the body as function of the distance to the skin, a significant ρ_{TOT}=ρ_{M1}+ρ_{M2} can be achieved, as desired.

(b) Another embodiment uses the principle of the ‘feeding magnet’. According to this principle, the magnetic elements need not to be electromagnets themselves, but may be made out of magnetizable material that is being magnetized by the presence of an electromagnet. See FIG. 12. An advantage of this is that the magnetic elements may be lighter, and therefore can achieve higher rotational speeds.

(c) Yet another preferred embodiment uses magnetic elements that have been composed of multiple small objects of magnetizable material that are rigidly connected to each other, in which the magnetizable material is composed of so-called ‘soft’ magnetic material, such as e.g. soft iron. Furthermore, each of these objects, that are thus rigidly connected and hence constitute one single magnetic element, is shaped in a geometrical shape that strongly deviates from a sphere or a cube, because the shape of each of these objects is such that its size (length) in one particular direction is at least 3 times as large as the size (width) in another direction. Examples of small objects fulfilling this requirement are, for example. oblong beams of which the length is at least 3 times as large as the width, and at least three times as large as the height.

Because of the shape of these small objects, the phenomenon called ‘shape anisotropy’ occurs in these small objects, i.e.: the magnetizability in the long direction (the ‘easy direction’) is much larger that in the other directions perpendicular to the easy direction.

This can be used, according to the principle of the ‘feeding magnet’ described above, to create a situation in which the total magnetic field remains virtually constant inside the tissues during rotation of the magnetic elements, despite the fact that the direction of magnetization in each individual small object may be perpendicular to the axis of rotation. See FIG. 13.

(d) Yet another preferred embodiment uses the principle of the ‘feeding magnet’ in the way described above at (c), but does not use shape anisotropy, but rather other forms of anisotropy, like ‘crystalline anisotropy’.

(e) the preferred embodiments (a) to (d) may be equipped with air bearings. Furthermore, airflow, pressurized air, or jets of air may be used to cause the rotation of the magnetic elements.

(f) the preferred embodiments (a) to (d) may be equipped as well with high-frequency vibrations to cause the rotation of the magnetic elements.

### ADDITIONAL PREFERRED EMBODIMENTS

Two systems consisting ((g) and (h)) of multiple arrays of rotating magnets are presented that do fulfil all requirements as listed above.

For each of the rotating magnetic wheels, the axis of rotation Ω of the magnetic element coincides with its axis of symmetry.

The first (g) of these two systems is a ‘sandwich’ configuration, i.e., the homogeneous conducting body R is located between two “walls” of rotating magnets.

The second system (h) is a single-sided configuration: all rotating magnets are located at one single side of the body R, and offers the additional advantage of a more sharply defined local maximum of the current density distribution.

Sandwich Configuration

(g) In the Sandwich configuration, the body (a homogeneous cube R) is ‘sandwiched’ by two sets of 150 rotating small wheels, in such a way that each set of wheels is placed parallel to one of two opposing sides of the cube R. See FIG. 14.

All wheels are rotating magnetic elements, made of a compressed mixture of iron powder and glue, thus creating an electrically non-conductive, soft ferro-magnetic material. Each wheel is a solid cylinder of 17.8 mm in diameter, and has a thickness of 10.5 mm. Each set of wheels is organized as 15 rows (counting along the x-axis) of wheels; each row containing 10 wheels (counting along the y-axis).

The heart-to-heart distances between two neighbouring wheels are 12.4 mm in the x-direction, and 18.6 mm in the y-direction. The soft ferro-magnetic material of the wheels is magnetized by an external, non-rotating, magnetic field that is produced by a fixed, non-rotating, set-up containing electromagnets and a ferromagnetic framework to guide the magnetic field lines into the rotating wheels. This non-rotating set-up (not depicted in the figures) is such that each wheel is subjected to an impressed magnetic field B_{applied}, of which the strength (ranging from 0 Tesla to 2 Tesla) is controlled by the currents in the coils of the electromagnets.

For all wheels, the direction of B_{applied} is parallel to the x-axis. At any given point in time, the strength of the impressed B-field B_{applied} is the same for all wheels, and homogeneous over the flat surface of each wheel. The distance between the surface of the box R and the round surfaces of the wheels is 13.5 mm.

FIG. 17 shows the magnetic field B_{onewheel} originating from the soft-magnetic material from a single wheel when subjected to an externally applied field B_{applied} of 2 Tesla (i.e., FIG. 17 shows the magnetic field after subtraction the applied field of 2 T).

Since the rotation vector Ω is parallel to the x-axis for each rotating magnet, the value of Bₚₐᵣ(x) is simply equal to the x-component of the field B_{onewheel} (x).

FIG. 17A shows the values of Bₚₐᵣ(x) on a (x,z)-plane near the single rotating wheel as a contour plot; the values printed near the contours are in mT.

The (x,z)-plane runs through the centre of the single wheel; the horizontal line separating FIG. 17A from FIG. 18A denotes the bottom surface of the cube R.

Single-Sided Configuration

(h) In this configuration (h), all magnetic wheels are placed at one single side of a homogeneous, oblong rectangular body Rblock.

The magnetic wheels are divided into two groups (see FIG. 15B), one group (group 1) between x=0 and x=100, and another group (group 2) at x>100.

The magnetization vector of the wheels in group 1 is pointing in exactly the opposite direction with respect to the magnetization vector of the wheels in group 2.

In order to obtain extra control over the location of the region of maximum current density, an extra layer of smaller rotating magnets has been added in this configuration.

Each large wheel rotates 3 times slower and has a Bₚₐᵣ(x) at its center that is 2 times weaker compared to its neighbouring small wheel.

Possible Application in the Fight Against Back Pain.

A possible application of the invention described above is to fight pain in the back, and more specifically the fight against chronic low back pain.

When designing the preferred embodiment of the invention for the specific application of fighting pain in the back, the precise location of the nerve fibers, as well as the specific conductivity distribution and the specific Σ(x) distribution in the tissues, due to the local anatomy, have to be taken into account. Since the Σ-values of muscle tissue are much higher than the Σ-values for fat or bone, the dorsal muscles, situated directly near and parallel to the spine, are suitable to function as storage locations of the quasi-static charge accumulations. These volumes of muscle tissue are located at each side of the spine, near the spine. Between the two volumes of muscle tissue, inside and between the dorsal vertebrae, the neuronal tissue is located that needs to be activated; for example, inside the Dorsal Root Ganglia. During a sudden reversal of the polarity of the magnetic field, these accumulated charges are displaced, and activate neurons in the process, i.e.: during the flowing of the electrical charge from the muscle tissue at one side of the spine into the muscle tissue at the other side of the spine, the neurons inside and near the dorsal root ganglia are activated.

In this way, specific activation of thick fibers inside and near the Dorsal Root Ganglia may be achieved in a spatially selective way, and therefore, according to the Gate Control theory, local pain mitigation may be achieved.

FIG. 16 shows a sketch of a situation in which the invention at hand is applied to fight pain in the back. See ‘explanation of the figures’ for further explanation.

The present invention is not limited to the above listing of preferred embodiments. Many variations and modifications are possible, all within the framework of the Conclusions defined below.

1. An apparatus for generating an electrical current field in the human body, comprising:
two or more rotatable magnetic elements for generating of a composite magnetic field, which magnetic elements are positioned such that each magnetic element is rotatable around an axis of rotation;
means for producing a rotating motion of the magnetic elements such that upon rotation of one or more of the magnetic elements the composite magnetic field remains substantially constant; and
means for disturbing the composite magnetic field, for altering the magnetic field entirely.

2. The apparatus according to claim 1, wherein the magnetic field which is generated by a magnetic element, is cylindrical symmetrical around an axis of symmetry.

3. The apparatus according to claim 1, wherein the axis of symmetry of the magnetic field produced by a rotating magnetic element coincides with the axis of rotation of that rotating magnetic element.

4. The apparatus according to claim 1, wherein the magnetic elements contain an electromagnet, a permanent magnet and/or magnetizable material.

5. The apparatus according to claim 1, wherein one or more magnetic elements consist of magnetizable material which is magnetized by the vicinity of a non-rotating electromagnet, permanent magnet and/or object of magnetizable material.

6. The apparatus according to claim 1, wherein the disturbing means comprise polarity reverse means to change polarity with a reversed frequency of one or more magnetic elements.

7. The apparatus according to claim 6, wherein the reverse polarity mean comprise an electrical switching devise which reverses the direction of the current flowing through the coil of an electromagnet.

8. The apparatus according to claim 1, wherein the rotation means comprise air thrust.

9. The apparatus according to claim 1, wherein the rotation means comprise high frequency oscillations.

10. The apparatus according to claim 1, wherein the rotation means comprise an electric motor.

11. The apparatus field according to claim 1, wherein the rotation frequency has a larger value than the reverse polarity frequency.

12. The apparatus field according to claim 1, wherein the polarity reversal frequency is lower than 3000 Hz.

13. The apparatus according to claim 11, wherein the polarity reversal frequency is lower than 1000 Hz.

14. The apparatus according to claim 1, wherein the magnet element has air bearings.

15. The apparatus according to claim 1, wherein one or more magnetic elements are composed of a number of oblong, beam-shaped small objects of magnetizable material, in which the shape of each of these objects is such that its size (length) in one particular direction is at least three times as large as the size (width) in another direction.

16. The apparatus according to claim 1, wherein one or more magnetic elements consist of a composition of small objects of magnetizable material, one or more of these small objects showing anisotropical magnetizable properties.

17. The apparatus according to claim 1, wherein the magnetic elements comprise a large number of relatively small wheels or discs of magnetizable material, which wheels or discs are rotated at a speed of more than 100.000 rpm.

18. The apparatus of claim 1, incorporated in a bed for patients or a backpack, wherein the apparatus is arranged close to the human skin, the electric current field being directed into or near the spine, more preferably inside the dorsal root ganglia, wherein the electrical charge for muscle tissue at one side of the spine flows into the muscle tissue at the other side of the spine, without inducing charge flow at or near the skin.

19. A method for generating of an electrical current field using an apparatus according to claim 1.

20. A method for selective neurostimulation using a device according to claim 1.

21. A method according to claim 19, wherein the electrical current field is directed into pain centers of shingles.
